(19) United States
(12) Patent Application Publication
(10) Pub. No.: US 2021/0244680 A1
(43) Pub. Date: Aug. 12, 2021

(54) WEARABLE LIPOSOMAL CANNABIDIOL TRANSDERMAL PATCH

(71) Applicants: Ghassan S. Kassab, La Jolla, CA (US); Ali Dabiri, San Diego, CA (US)

(72) Inventors: Ghassan S. Kassab, La Jolla, CA (US); Ali Dabiri, San Diego, CA (US)

(21) Appl. No.: 17/172,235

(22) Filed: Feb. 10, 2021

Related U.S. Application Data
(60) Provisional application No. 62/972,624, filed on Feb. 10, 2020.

(51) Int. Cl. A61K 9/70; A61K 31/05; A61K 9/00

(52) U.S. Cl. CPC A61K 9/7084; A61K 9/0021; A61K 31/05

(57) ABSTRACT

A wearable patch for the transdermal delivery of cannabidiol (CBD). Cannabidiol is delivered in in the form of a CBD mixture which comprises CBD oil loaded into liposomes. The CBD mixture can be delivered without the use of dermal enhancers. A transdermal patch is preloaded with the CBD mixture and placed upon the skin to penetrate the dermis layer, where the CBD mixture will diffuse into the interstitial body fluid. The patch comprises microneedles and the CBD mixture which is preloaded into or on the needles depending on the type of microneedles used.

### PRIORITY

The present patent application is related to, and claims the priority benefit of, U.S. Provisional Patent Application Ser. No. 62/972,624, filed on Feb. 10, 2020, the contents of which are hereby incorporated by reference in their entirety into this disclosure.

### BACKGROUND

Over 100 million Americans are living with chronic pain, and pain is the most common reason that patients seek medical attention. Despite the prevalence of pain, the practice of pain management and the scientific discipline of pain research are relatively new fields compared to the rest of medicine—contributing to a twenty-first century dilemma for health care providers asked to relieve suffering in the “Fifth Vital Sign” era (1). The assessment and treatment of chronic pain will continue to be one of the most common functions of a health care provider. To move beyond an over reliance on opioid medications, the addiction and pain research communities must unite with chronic pain patients to increase the evidence base supporting non-opioid analgesic strategies (1).

Cannabidiol (CBD) oil is one of more than 120 compounds (2) called cannabinoids. Many plants contain cannabinoids, but people most commonly link them to cannabis. Unlike other cannabinoids, such as tetrahydrocannabinol (THC), CBD does not produce a euphoric “high” or psychoactive effect. This is because CBD does not affect the same receptors as THC. People have used CBD for thousands of years to treat various types of pain, but the medical community has only recently begun to study the benefits. The human body has an endocannabinoid system (ECS) that receives and translates signals from cannabinoids. It produces some cannabinoids of its own, which are called endocannabinoids. The ECS helps to regulate functions such as sleep, immune-system responses, and pain.

Different cannabis plants, often called hemp or marijuana, contain different levels of chemical compounds. How people breed the plant affects the CBD levels. Most CBD oil comes from industrial hemp, which usually has a higher CBD content than marijuana. Makers of CBD oil use different methods to extract the compound. CBD oil comes in many different strengths, and people use it in various ways.

### Benefits

Hammell et al., (3) used an animal model to determine if CBD could help people with arthritis manage their pain. Researchers applied a topical gel containing CBD to rats with arthritis for 4 days. Their findings were a significant drop in inflammation and signs of pain, without additional side effects. People using CBD oil for arthritis may find relief from their pain, but more human studies need to be done to confirm these findings. Multiple sclerosis (MS) is an autoimmune disease that affects the entire body through the nerves and brain. Muscle spasms are one of the most common symptoms of MS. These spasms can be so great they cause constant pain in some people. It is reported (4) that short-term use of CBD oil could reduce the levels of spasticity a person feels. The results are modest, but many people reported a reduction in symptoms. More studies on humans are needed to verify these results. The same report studied CBD use for general chronic pain. Researchers compiled the results of multiple systematic reviews covering dozens of trials and studies. Their research concluded that there is substantial evidence that cannabis is an effective treatment for chronic pain in adults. Other studies (5, 6) support these results. This research suggests that using CBD can reduce pain and inflammation. The researchers also found that subjects were not likely to build up a tolerance to the effects of CBD, so they would not need to increase their dose continually. They noted that cannabinoids, such as CBD, could offer helpful new treatments for people with chronic pain.

There are other promising applications for CBD like smoking cessation (7) and drug withdrawal (8), treating seizures and epilepsy (9), anxiety (10) treatment, reducing some of the effects of Alzheimer's (11), as shown by initial research, antipsychotic effects on people with schizophrenia (12), future applications in combating type 1 diabetes (13) and cancer (14,15). Although more research is required to confirm some uses of CBD oil, it is shaping up as a potentially promising and versatile treatment.

The U.S. Food and Drug Administration (FDA) does not regulate CBD for most conditions. As a result, dosages are currently open to interpretation, and people should exercise caution. Under the Controlled Substances Act (CSA), CBD is currently a Schedule I substance because it is a chemical component of the cannabis plant. FDA has approved three drugs based on cannabinoids. Dronabinol, the generic name for synthetic Delta9-THC, is marketed under the trade name of Marinol® and its FDA-approved uses are to counteract the nausea and vomiting associated with chemotherapy and to stimulate appetite in AIDS patients affected by wasting syndrome. A synthetic analog of D9-THC, nabilone (Cesamet®), is prescribed for similar indications. Both dronabinol and nabilone are given orally and have a slow onset of action. In July 2016, the FDA approved Syndros®, a liquid formulation of dronabinol, for the treatment of patients experiencing chemotherapy-induced nausea and vomiting who have not responded to conventional antiemetic therapies. The agent is also indicated for treating anorexia associated with weight loss in patients with AIDS. Two additional cannabinoid-based medications have been examined by the FDA. Nabiximols (Sativex®) is an ethanol cannabis extract composed of D9-THC and CBD in a one-to-one ratio. Nabiximols is administered as an oromucosal spray and is indicated in the symptomatic relief of multiple sclerosis and as an adjunctive analgesic treatment in cancer patients (16). As of September 2016, nabiximols has been launched in 15 countries, including Canada, Germany, Italy, Spain, the United Kingdom, and has been approved in additional 12 countries, but not thus far in the United States. In response to the urgent need expressed by parents of children with intractable epilepsy, FDA approved Epidiolex cannabidiol oral solution for the treatment of seizures associated with two rare and severe forms of epilepsy, Lennox-Gastaut syndrome and Dravet syndrome, in patients two years of age and older (17). This is the first FDA-approved drug that contains a purified drug substance derived from marijuana, CBD. It is also the first FDA approval of a drug for the treatment of patients with Dravet syndrome. The patient information leaflet for Epidiolex cautions that there is a risk of liver damage, lethargy, and possibly depression and thoughts of suicide, but these are also true of other treatments for epilepsy.

The FDA is working to answer questions about the science, safety, and quality of products containing cannabis and cannabis-derived compounds, particularly CBD. It is working towards public hearing to obtain scientific data and information about the safety, manufacturing, product quality, marketing, labeling, and sale of products containing cannabis or cannabis-derived compounds (18).

Side Effects

There are some possible side effects, although most people tolerate CBD oil well. The most common side effects include: tiredness, diarrhea, changes in appetite, weight gain or weight loss (19). In addition, using CBD oil with other medications may make those medications more or less effective (19). The scientists have yet to study some aspects of CBD, such as its long-term effects on hormones. Further long-term studies will be helpful in determining any side effects CBD has on the body over time (19). CBD might also interfere with an enzyme called cytochrome P450 complex. This disruption can affect the liver's ability to break down toxins, increasing the risk of liver toxicity (19). On the other hand, CBD oil does show a lot of potential for pain relief. Anecdotal evidence suggests that it can be used to help manage chronic pain in many cases. CBD oil is especially promising due to its lack of intoxicating effects and a possible lower potential for side effects than many other pain medications.

### Way to Use CBD for Pain-Management

As the CBD market continues to develop, its products have become more sophisticated, offering more value and new ways to use CBD. While there are certainly a wide variety of ways to use CBD, each offers certain advantages and disadvantages. This leads many to wonder, “what's the best way to use CBD?”

There are important factors CBD users should consider to determine which method of consumption is best for them. Some of these factors include: 1) The method's bioavailability rate or the amount of CBD that enters the bloodstream, 2) The maximum concentration of CBD delivered from the method, 3) The average cost versus value of the method. The top methods are:

1. CBD Vape Pens and E-Liquid

Aside from the advantages offered by traditional vaping (alternative to smoking, relaxing, enjoyable flavors, etc.), vaporizing allows the CBD to enter the bloodstream directly through the lungs. As a result, a higher bioavailability of CBD is delivered to the body, more quickly and more effectively than if it had gone through the body's digestive tract. Vaping CBD is one of the most popular methods of consuming CBD, and the first method on our list. The problem might be the potential side effect on the lung for long term users.

2. CBD Oil Tinctures

CBD tinctures are made by extracting cannabinoids from the hemp plant, then infusing the cannabinoids with a liquid solvent, such as alcohol or apple cider vinegar. As a liquid concentrate, CBD tinctures are administered sublingually under the tongue, where the CBD oil is then absorbed into the salivary glands under the tongue. While the saliva on the tongue does breakdown a portion of the CBD, it enters the bloodstream more quickly and does not get filtered out by the body's digestive tract.

3. CBD Capsules and Softgels

While vaporizing CBD provides higher bioavailability and CBD tinctures provide quicker results, CBD capsules provides important benefits that the other methods may not which is discretion and accurate, consistent dosages. With capsules, one does not have to deal with the stigma of vaping or the uninviting flavors of tinctures, but, of course, capsules do have their downfalls. For example, capsules take longer to take effect and deliver less bioavailability of CBD to the body because our bodies take longer to break down a capsule, and a capsule will also go through most of our bodies filtration systems. Still, it is a great option for many users, and to offset the decrease in bioavailability, one can always take a higher concentrated capsule.

4. CBD Patches

CBD patches are an excellent way to maximize the powerful healing effects of cannabidiol. Applied to the skin, they are designed to deliver CBD transdermally, which bypasses the digestive system and makes it more bioavailable than some other methods. As your body heat warms the patch, small amounts of CBD are released through the skin to give you a consistent and ongoing dose. They are portable, discreet, and can be cut with scissors to customize their potency to your needs. Ideal for neuropathic pain, fibromyalgia, seizure control, and a host of other conditions. There are several CBD patches available in the market. One of the commercially available patches lasts up to 96 hours and contains 40 mg of CBD. Not all the CBD amounts will be absorbed due to decreasing CBD gradients across the skin over the residence time. The hourly average comes out to be about 0.2 μl which is not sufficient for rapid pain relief.

Aside from the methods we have already discussed, there are numerous other ways to use CBD such as:CBD Edibles and Beverages, CBD Topicals and Lotions, High-CBD Cannabis Strains.

### BRIEF SUMMARY

The disclosure provides for a device and method of applying CBD oil transdermally via a patch.

Enough CBD must be absorbed from the patch to generate the desired concentration in the blood to reduce the pain in a reasonable time which is not usually the case due to limited CBD concentration gradient across the skin, especially for the rapid pain relief cases to substitute the pain medications. Microneedles are proposed to be used in these situations.

Microneedle arrays are minimally invasive devices that can be used to bypass the stratum corneum barrier and thus accessing the skin microcirculation and achieving systemic delivery by the transdermal route for drug delivery. Microneedles (MN) (hundreds of microns in length up to 1000 MNcm⁻²) with diverse geometries have been produced from silicon, metal, and polymers using various microfabrication techniques. MNs have been prepared using chemical isotropic etching, injection molding, reactive ion etching, surface/bulk micromachining, micro-molding, and lithography-electroforming-replication. MNs are applied to the skin surface and pierce the epidermis (devoid of nociceptors) painlessly without skin infection, creating microscopic holes through which drugs diffuse to the dermal microcirculation. MNs can be made long enough to penetrate to the dermis layer but are typically short and narrow enough to avoid stimulation of dermal nerves and puncture dermal blood vessels (20). MNs are classified as solid, hollow, and polymeric depending on the application. Solid MNs puncture skin prior to application of a drug loaded patch or are pre-coated with drug prior to insertion. Hollow bore microneedles allow diffusion or pressure-driven flow of drugs through a central lumen. The polymeric MNs are either of dissolved type or hydrogel-forming. The dissolved MNs release their drug payload as they dissolve in the skin layers and are generally a biocompatible polymer. The skin insertion of the array is followed by dissolution of the MNs tips upon contact with skin interstitial fluid. The drug is then released over time. The hydrogel-forming MNs take up interstitial body fluids (IBL) from the tissue, inducing diffusion of the drug located in a patch through the swollen micro projections (21). The amount of swelling can be controlled by adding different agents. Hydrogel forming MNs are removed intact from skin, leaving no measurable polymer residue behind. They cannot be reused since there is a potential of getting softer. MN polymers are drawing increasing attention because of their excellent biocompatibility, biodegradability, low toxicity and strength/toughness. They are easy to fabricate and cost-effective. One candidate material could be aqueous blends containing 15% w/w poly (methylvinylether/maelic acid) (PMVE/MA) and 7.5% w/w poly(ethyleneglycol) 10,000 (PEG) to fabricate MN. MN were crosslinked by esterification with poly(ethyleneglycol) to enable the swelling and prevent in-skin dissolution (22). It is robust and not only punctures the stratum corneum of human skin in vivo, but also protrudes quite deeply into the underlying viable epidermis and upper dermis with relatively low insertion force of 0.03 N(newton)/MN. The height of microneedles is about 600 μm with about 500 μm extended into the skin. The interspacing of MN at the base is about 300 μm with the width at the base of about 300 μm. The MN can be fabricated by laser based micro-molding technique. For example, an array of 11×11 needles with these dimensions takes about five minutes to be machined at ambient temperature using current technology. The substrate can ideally possess some degree of flexibility to accommodate the irregular topography of the skin surface due to macroscopic curvature of different body regions to prevent break of MN during insertion.

Previous studies have shown that 83% of proteins found in serum are also in interstitial body fluid (IBF), but 50% of proteins in IBF are not in serum, suggesting that interstitial body fluid may be a source of unique biomarkers as well as biomarkers found in blood (23,24). Skin is the most accessible organ and therefore a source of IBF containing biomarkers. Most of skin's IBF is in dermis (25), which comprises a network of collagen and elastin fibers surrounded by extracellular matrix that limits IBF flow due to binding and tortuosity. It is estimated that ˜70 wt % of human dermis comprising IBF (26). There are several mechanisms of IBF collection into MN including diffusion, capillary and osmotic actions (27).

A primary objective of the present method and apparatus is to provide new systems and technique to use CBD more readily and effectively than the current methods via interstitial body fluid. The device features will be 1) use of polymer microneedles, 2) minimally invasive, 3) fast response to pain reduction after the application (Point of Care), 4) Bio-material compatibility, 5) Ease of manufacturing, 6) Moderate cost, 7) Long shelf life, 8) Ease of application/removal, 9) No skin side effect like irritation or allergic reactions to the microneedle, and 10) Minimum environmental contamination of microneedle before application or after removal.

Recent progress indicates the possibility of 1-10 μl of IBF within 20 min though MN. As noted above, the hourly average of CBD release in a patch is about 0.2 μl. It is a good assumption that the concentration of CBD in the interstitial body fluid is about the same as in the blood. Therefore, microneedle patches can increase the CBD concentration by a factor of at least 10 for the same patch residence time, making it feasible for those individuals that need rapid pain relief.

Another advantage of this method is that there is no need for enhancers, e.g., DMI (1,3-Dimethyl-2-imidazolidinone) which is a natural dermal penetrant that enables CBD to be more easily absorbed into the skin.

The CBD oil will be mixed with liposome to enhance bioavailability of the CBD. producing a CBD mixture. A liposome has an aqueous solution core surrounded by a hydrophobic membrane in the form of a lipid bilayer. A liposome can be loaded with hydrophobic molecules like CBD oil. To deliver the molecule to a site of action, the lipid bilayer can fuse with other bilayers, thus delivering the liposome contents (CBD) to increase bioavailability.

The present disclosure includes disclosure of systems and methods to produce and use the same, as shown and/or described herein.

The present disclosure includes disclosure of needle devices and methods to produce and use the same, as shown and/or described herein.

The present disclosure includes disclosure of delivery devices and methods to use the same, as shown and/or described herein.

The present disclosure includes disclosure of microneedle devices and methods to produce and use the same, as shown and/or described herein.

The present disclosure includes disclosure of a microneedle device, comprising an adhesive layer, and a microneedle substrate adhered to the adhesive layer, and a) wherein the microneedle substrate has a plurality of microneedles coupled thereto, or b) wherein the microneedle substrate further comprises the plurality of microneedles. The microneedle device can be firmly attached to the skin by adhesive layer. The microneedle device comprises a release liner, where the release liner covers the microneedle device during storage and prior to use, to avoid potential contamination of microneedle device. The release liner is removed before use.

The present disclosure includes disclosure of a microneedle device, comprising a membrane (which, along with an adhesive, can be considered as an “adhesive layer”). A microneedle substrate adhered thereto (adhered to the membrane, which, along with the adhesive, can be the adhesive layer), and a) wherein the microneedle substrate has a plurality of microneedles coupled thereto, or b) wherein the microneedle substrate further comprises the plurality of microneedles.

The present disclosure includes disclosure of a microneedle device, forming part of a system, the system further comprising at least CBD mixture, the wells configured to hold CBD mixture.

The present disclosure includes disclosure of a method to use a microneedle device, comprising the steps of placing a microneedle device of the present disclosure upon skin of a wearer so to cause at least part of a plurality of microneedles of the microneedle device to enter a dermis of the skin to deliver CBD mixture to interstitial body fluid, and removing the microneedle device from the skin after a period of time elapses, said period of time being enough time to permit microneedle to release CBD mixture to interstitial body fluid.

In an exemplary embodiment, a method of using a transdermal patch to treat a patient with acannabidiol (CBD) comprises the steps of loading the device with a CBD mixture, wherein the CBD mixture comprises CBD oil and liposomes; applying the patch to the skin; and allowing the CBD mixture to penetrate into the skin.

In an alternate embodiment, a method of using a transdermal patch to treat a patient with CBD also comprises the steps of removing a release liner from the patch before applying the patch to the skin.

In an alternate embodiment, a method of using a transdermal patch to treat a patient with CBD also comprises the step of allowing the CBD mixture to penetrate the skin is pressure driven.

In an alternate embodiment, a method of using a transdermal patch to treat a patient with CBD the step of allowing the CBD mixture to penetrate the skin does not utilize dermal penetration enhancers.

In an alternate embodiment, a method of using a transdermal patch to treat a patient with CBD also comprises the step of releasing the CBD mixture into the interstitial body fluid.

In an alternate embodiment, a method of using a transdermal patch to treat a patient with CBD also comprises the step of allowing the CBD mixture to penetrate the skin releases CBD mixture at a rate greater than 0.2 μl of CBD mixture per hour.

In an alternate embodiment, of a method of using a transdermal patch to treat a patient with CBD, the step of applying the patch to the skin also comprises the step of using an at least one microneedle on the patch to to puncture the stratum corneum, the viable epidermis, and the dermis, so that when the patch is positioned upon the skin, a tip of the at least one microneedle are positioned within the dermis.

In an exemplary embodiment a microneedle device, comprises: a membrane having an adhesive thereon; a microneedle substrate adhered to the membrane using the adhesive; a release layer positioned upon the microneedle device to protect the microneedle substrate; wherein the microneedle substrate has a plurality of microneedles coupled thereto; and a CBD mixture comprising CBD oil within liposomes is loaded onto or into the microneedle device.

In an alternate embodiment of a microneedle device, the microneedles are solid and coated with the CBD mixture. In an alternate embodiment of a microneedle device, the microneedles are hollow and are connected to a pressure mechanism configured to force CBD mixture out of the needles. In an alternate embodiment of a microneedle device, the plurality of microneedles comprises microneedle tips configured to dissolve in interstitial body fluid.

In an alternate embodiment, the microneedle device forms part of a system, the system further comprising at least one of the following: a drug container having wells defined therein, the wells configured to hold CBD mixture; and/or a delivery device.

In an alternate embodiment of a microneedle device, the plurality of microneedles is at least partially coated with the CBD mixture.

In an exemplary embodiment, a method of using a microneedle device for delivery of CBD, comprises the steps of: placing a microneedle device of the present disclosure upon skin of a wearer so to cause at least part of a plurality of microneedles of the microneedle device to enter a dermis of the skin; and removing the microneedle device from the skin after a CBD mixture enters the interstitial body fluid from the plurality of microneedles, the CBD mixture comprising CBD oil mixed with liposomes.

In an alternate embodiment of a method of using a microneedle device for delivery of CBD the method includes the step of removing the microneedle device from a drug container, wherein the drug container comprises wells filled with CBD mixture.

In an alternate embodiment of a method of using a microneedle device for delivery of CBD further comprises the step of driving the CBD mixture into the interstitial body fluid via a pressure mechanism.

In an alternate embodiment of a method of using a microneedle device for delivery of CBD, the CBD mixture enters the interstitial body fluid from the plurality of microneedles without the use of dermal penetrant enhancers.

In an alternate embodiment of a method of using a microneedle device for delivery of CBD the CBD mixture is introduced into the interstitial body fluid at a rate of greater than 0.2 μl per hour.

In an alternate embodiment of a method of using a microneedle device for delivery of CBD, the method further comprises the step of allowing the liposome carrying the CBD oil to fuse with bilayers within the patient to deliver the CBD oil.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a delivery device used to deliver CBD mixture, according to an exemplary embodiment of the present disclosure;

FIG. 2 shows a microneedle device having a plurality of hollow needles, used to deliver CBD mixture, according to an exemplary embodiment of the present disclosure;

FIG. 3 shows a microneedle device applied to the skin, according to an exemplary embodiment of the present disclosure;

FIG. 4 microneedle device and release liner according to an exemplary embodiment of the present disclosure; and

FIG. 5 shows a Drug container according to an exemplary embodiment of the present disclosure.

As such, an overview of the features, functions and/or configurations of the components depicted in the various figures will now be presented. It should be appreciated that not all of the features of the components of the figures are necessarily described and some of these non discussed features (as well as discussed features) are inherent from the figures themselves. Other non-discussed features may be inherent in component geometry and/or configuration.

Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

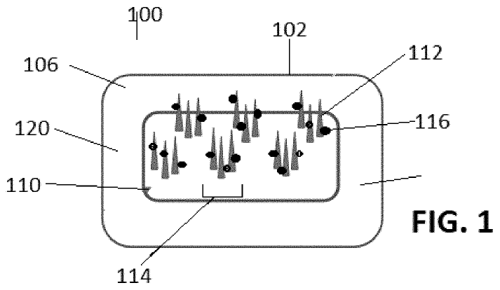

In an embodiment of the invention a transdermal patch 100 as shown in FIG. 1 is utilized to dispense drugs. In this embodiment, the drug dispensed is CBD oil, specifically a CBD mixture 116 comprising CBD oil mixed with liposomes. The patch 100 provides for the release of a sufficient amount of drug at reasonable residence time such that the desired symptom effectively treated.

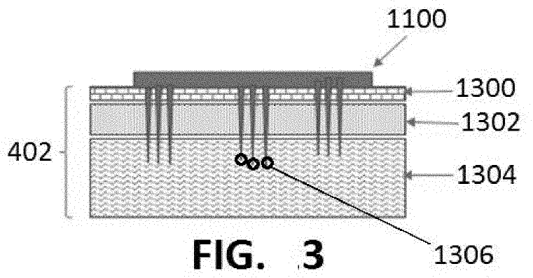

An exemplary patch/microneedle device 100 (also referred to herein as a transdermal patch) of the present disclosure is shown in FIG. 1. As shown therein, patch 100 comprises a membrane 102, and an adhesive layer 106 present upon membrane 102 (an adhesive being applied to membrane 102), whereby adhesive 106 facilitates adhesion of microneedle substrate 110 to membrane 102 and adhesion of patch 100 to a wearer's skin 402 shown in FIG. 3.

An exemplary adhesive layer 106 of the present disclosure can comprise any number of suitable adhesives, such as bio adhesives (Duro-TAK 387-2510/87-2510 from Henkel, for example) or other materials which is/are mixed with sodium carboxymethyl cellulose (NaCMC) or other materials, resulting in a total adhesive layer 106 thickness of 100 to 150 μm, or thicker or thinner.

Membranes 102 of the present disclosure essentially exist as a backing film on an opposite side of adhesive layer 106 used to adhere to the skin of a wearer. The total thickness of the membrane 102 can be 200 to 300 μm, or thicker or thinner.

The patch 100 comprises an adhesive layer 106 on top of a membrane 102 such that adhesive 106 is present between membrane 102 and a microneedle substrate 110 to adhere microneedle substrate 110 to membrane 102. Preferably, the adhesive layer 106 covers the entire membrane 102. Microneedles 112 can be arranged upon microneedle substrate 110 in microneedle groups 114 as desired, whereby each microneedle group 114 comprises a plurality of microneedles 112. Preferably, the microneedle substrate 110 will have a smaller area than the adhesive layer such that a border 120 is formed around the microneedle substrate that comprises only the adhesive layer 106 and the membrane 102.

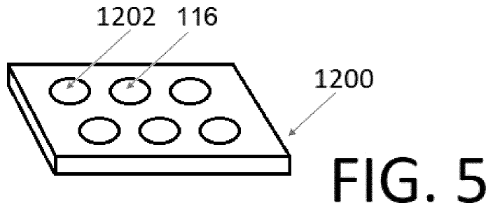

In an embodiment of a system for dosing CBD mixture, the system also comprises a drug container. Said groups 114 of microneedles 112 can be arranged about microneedle substrate 110 so to correspond with locations of wells 1202 defined within a corresponding drug container 1200, whereby CBD mixture 116 is present within said wells 1202 of drug container 1200, as in FIG. 5. The microneedles can take up CBD mixture from the wells of the drug container.

In an embodiment, the drug delivered is CBD oil or more specifically, CBD oil mixed with liposome creating a CBD mixture 116.

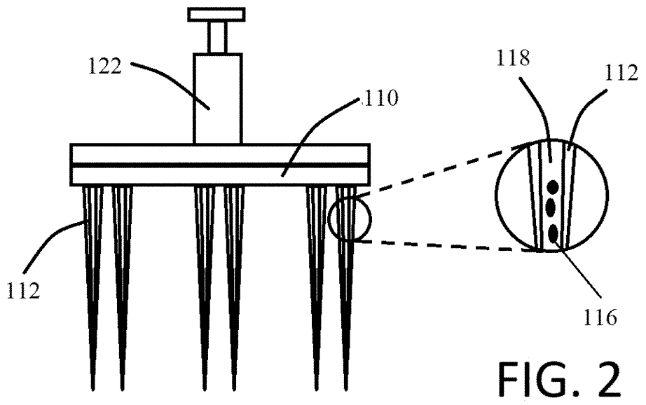

The microneedles may be of any type, solid, hollow or polymeric. As represented in FIG. 1, the drug, CBD mixture 116, is preloaded onto or into the microneedles. Solid MNs puncture skin thereby introducing the pre-coated CBD mixture 116 into the body. As shown in FIG. 2, hollow bore microneedles allow diffusion or pressure-driven flow of drugs through a central lumen 118. They may be attached to a pressure mechanism 122 allowing them to force out CBD mixture, as in FIG. 3. The polymeric MNs are either of dissolved type or hydrogel-forming. The dissolved MNs release their CBD mixture payload as they dissolve in the skin layers and are generally a biocompatible polymer. The skin insertion of the array is followed by dissolution of the MNs tips upon contact with skin interstitial fluid. The CBD mixture is then released over time. The hydrogelforming MNs releases CBD mixture to interstitial body fluids (IBF) through diffusion.

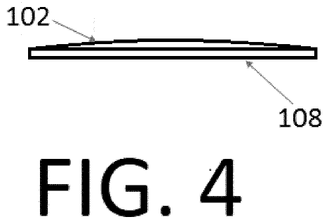

To protect and maintain sterility of microneedle device 100, a release liner 108 can be used to cover the side of microneedle device 100 having the plurality of microneedles, such as shown in FIG. 4. When release layer is removed, a plurality of microneedles 112 is revealed, such as shown in FIG. 1.

Like the other embodiments, the patch may also comprise a release liner 108. A release liner may cover the patch during storage and prior to use, so to avoid potential contamination. Release liner 108 is removed before use to expose microneedles. Release liners 108 can have a thickness between 50 to 70 μm, or larger or smaller.

Microneedle devices 100 of the present disclosure ideally include the fewest number of microneedles 112 necessary in order to deliver a dosage of CBD mixture effective to treat pain of the wearer of microneedle device 100. For example, and as shown in FIG. 1, each group 114 of microneedles 112 contains three microneedles 112, and with six groups 114 (an exemplary number of groups), that would be eighteen microneedles 112 in total. Other microneedle devices 100 may include any desired number of groups 114 of microneedles 112, with any desired number of microneedles 112 per group 114, such as a) six groups 114 of three microneedles 112 each (so eighteen total microneedles 112), b) six groups 114 of six microneedles 112 each (so thirty-six total microneedles 112), c) four groups 114 of four microneedles 112 each (so sixteen total microneedles 112), d) six groups 114 of twelve microneedles 112 each (so seventy-two total microneedles 112), etc.

In some embodiments of microneedle devices 100 of the present disclosure, microneedle devices 100 comprises a microneedle substrate 110 which is formed as part of an overall unit with microneedles 112, or which is coupled to microneedles 112 to help complete an embodiment of the microneedle device 100 that can withstand the desired uses as referenced herein. Substrate 1106, as referenced herein, can be relatively flexible so to accommodate the irregular topography of the surface of the skin 402 due to macroscopic curvature of different body regions to prevent breakage of microneedles 112 during insertion. As shown in FIG. 1, microneedle substrate 110 can be adhered to membrane 102 on one side and microneedle substrate 110 on another, using adhesive 106, as may be desired.

FIG. 3 shows several layers of skin 402, including the stratum corneum 1300, viable epidermis 1302, and dermis 1304 containing interstitial body layer (IBL), from the outside moving inward. When microneedle device 100 is positioned upon the skin 402 (first the stratum corneum 1300), microneedle device 100 can then be pressed in the direction of skin 402 to cause microneedles 112 to puncture the stratum corneum 1300, the viable epidermis 1302, and the dermis 1304, in that order, so that when completely positioned upon the skin 402, microneedle device 100 contacts the skin 402, and the relative tips of microneedles 112 are positioned within the dermis 1304. This allows the CBD mixture to be introduced into the interstitial body layer.

### Method of Use for Transdermal CBD Patch

An exemplary embodiment of using a CBD patch comprises the steps of: 1) preloading the device with CBD mixture; 2) removing a release liner, 3) applying the patch to the skin; and 4) allowing the CBD mixture to penetrate the skin to the dermis layer. The patch may be removed after use.

The step of preloading the device with CBD mixture, can comprise the step of coating CBD mixture on the needles, or loading CBD mixture into the needles. This step may be performed using the drug container having wells filled with CBD mixture. The microneedles will align with the wells and the two elements can be mated to coat the needles with the CBD mixture.

The step of applying the patch to the skin comprises the step of piercing the skin with the microneedles attached to the patch. The patch is secured in place with the adhesive. Preferably, the tips of the microneedles are located in the dermis layer of the skin after piercing.

The step of allowing the CBD mixture to penetrate the skin to the dermis layer may comprise the step of leaving the patch on the skin of the wearer for a reasonable residence time.

The step of allowing the CBD mixture to penetrate the dermis layer, may comprise the step of allowing the CBD mixture to diffuse into the dermis or/and through dissolving the microneedles.

Ultimately, the type of microneedles used will determine the exact mechanism of drug introduction. For example, where the microneedles are solid, the CBD mixture may directly enter the interstitial body fluid. Hollow needles may use a pressure mechanism to drive the CBD mixture from a reservoir out of the needles and into the interstitial body fluid. In some embodiments, diffusion may allow CBD mixture within the path to flow out of the hollow needles. In another embodiment, the microneedles may dissolve releasing the CBD mixture. Alternatively, the microneedles may swell, and CBD mixture may flow from the microneedles into the interstitial body fluid.

The patch can be used to treat pain, chronic or acute, inflammation, or any other malady treatable with CBD.

While various embodiments of devices for dosing CBD transdermally and methods for the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCES

1. Tompkins, D. A., Hobelmann, J. G. and Compton, P., Providing chronic pain management in the “Fifth Vital Sign” Era: Historical and treatment perspectives on a modern-day medical dilemma, Drug Alcohol Depend, 173(Suppl 1): S11-S21, 2107.
2. Morales, P., Hurst, D. P., and Reggio, P. H., Molecular Targets of the Phytocannabinoids-A Complex Picture, Prog Chem Org Nat Prod., 103: 103-131, 2017.
3. Hammell, D. C. et al., Transdermal cannabidiol reduces inflammation and pain-related behaviors in a rat model of arthritis, Eur J Pain. 20, 6: 936-948, 2016.
4. The Health Effects of Cannabis and Cannabinoids: The Current State of Evidence and Recommendations for Research. National Academies of Sciences, Engineering, and Medicine; Health and Medicine Division; Board on Population Health and Public Health Practice; Committee on the Health Effects of Marijuana: An Evidence Review and Research Agenda. Washington (DC): National Academies Press (US); Jan. 12. 2017.
5. Xiong, W., Cannabinoids suppress inflammatory and neuropathic pain by targeting α3 glycine receptors, J Exp Med.; 209, 6: 1121-1134, 2012.
6. Bruni, N., et al., Cannabinoid Delivery Systems for Pain and Inflammation Treatment, Molecules, 23, 2478,1-25, 2018.
7. Morgan C J et al., Cannabidiol reduces cigarette consumption in tobacco smokers: preliminary findings, Addict Behav. 38, 9:2433-6, 2013.
8. Hurd, Y. L., et. al., Early Phase in the Development of Cannabidiol as a Treatment for Addiction: Opioid Relapse Takes Initial Center Stage, Neurotherapeutics, 12, 4: 807-815, 2015.
9. Devinsky, O. et. al., Cannabidiol: Pharmacology and potential therapeutic role in epilepsy and other neuropsychiatric disorders, Epilepsia. 55, 6: 791-802, 2014.
10. Blessing E. M. et. al., Cannabidiol as a Potential Treatment for Anxiety Disorders, Neurotherapeutics. 12, 4: 825-836, 2015.
11. Cheng, D. et.al., Long-Term Cannabidiol Treatment Prevents the Development of Social Recognition Memory Deficits in Alzheimer's Disease Transgenic Mice, in Journal of Alzheimer's disease: JAD 42, 4, 2014.
12. Zuardi, A W., A critical review of the antipsychotic effects of cannabidiol: 30 years of a translational investigation. Curr Pharm Des.18, 32:5131-40, 2012
13. Lehmann C. et. al., Experimental cannabidiol treatment reduces early pancreatic inflammation in type 1 diabetes, Clin Hemorheol Microcirc., 64, 4:655-662, 2016.
14. Massi, P. et. al., Cannabidiol as potential anticancer drug, Br J Clin Pharmacol. 75, 2: 303-312, 2013.
15. Blasco-Benito, S., et.al., Appraising the “entourage effect”: Antitumor action of a pure cannabinoid versus a botanical drug preparation in preclinical models of breast cancer, Biochemical Pharmacology, 157, 285-293, 2018.
16. Pertwee, R. G., Targeting the endocannabinoid system with cannabinoid receptor agonists: Pharmacological strategies and therapeutic possibilities. Philosophical Transactions of the Royal Society of London B: Biological Sciences, 367, 1607:3353-3363, 2012.
17. FDA, For Immediate Release: Jun. 25, 2018.
18. FDA, Scientific Data and Information about Products Containing Cannabis or Cannabis-Derived Compounds; Public Hearing, May 31, 2019.
19. Iffland, 'K., and Grotenhermen, F., An Update on Safety and Side Effects of Cannabidiol: A Review of Clinical Data and Relevant Animal Studies, Cannabis Cannabinoid Res., 2, 1:139-154, 2017.
20. Donnelly, R. F. et al, Optimization and Characterization of Polymeric Microneedle Arrays Prepared by a Novel Laser-Based Micro-molding Technique, Pharm, 28, 41-57, 2011.
21. Larraneta, E., Lutton, R. E., Woolfson, A. D., Donnelly R. F., Microneedle arrays as transdermal and intradermal drug delivery systems: Materials science, manufacture and commercial development, Materials Science and Engineering R, 104,1-32, 2016.
22. Donnelly RF,′ Singh T R, Garland M J, Migalska K, Majithiya R, McCrudden C M, Kole P L, Mahmood T M, McCarthy H O, Woolfson A D, Hydrogel-Forming Microneedle Arrays for Enhanced Transdermal Drug Delivery, Adv Funct Mater., 22, 23, 4879-4890, 2012.
23. Kool J, et al. Suctionblister fluid as potential bodyfluid for biomarkerproteins. Proteomics 7, 3638-3650, 2007.
24. Muller A C, et al. A comparative proteomic study of human skin suction blister fluid from healthy individuals using immunodepletion and iTRAQ labeling. J Proteome Res 11, 3715-3727, 2012.
25. Skin anatomy. Available at https://emedicine.medscape.
26. Aukland K, Nicolaysen, Interstitial fluid volume: Local regulatory mechanisms. Physiol Rev 61, 556-643, 1981.
27. Samanta, P. P., and Prausnitza, M. R., Mechanisms of sampling interstitial fluid from skin using a microneedle patch, PNAS, 115, 2018.

1. A method of using a transdermal patch to treat a patient with cannabidiol (CBD) comprising the steps of:
loading the patch with a CBD mixture, wherein the CBD mixture comprises CBD oil and liposomes;
applying the patch to the skin; and
allowing the CBD mixture to penetrate the skin.

2. The method of using a transdermal patch to treat a patient with CBD of claim 1, further comprising the steps of:
removing a release liner from the patch before applying the patch to the skin.

3. The method of using a transdermal patch to treat a patient with CBD of claim 1,
wherein the step of allowing the CBD mixture to penetrate the skin does not utilize dermal penetration enhancers.

4. The method of using a transdermal patch to treat a patient with CBD of claim 1,
wherein the step of allowing the CBD mixture to penetrate the skin comprises the step of releasing the CBD mixture into the interstitial body fluid.

5. The method of using a transdermal patch to treat a patient with CBD of claim 1, wherein the step of allowing the CBD mixture to penetrate the skin releases CBD mixture at a rate greater than 0.2 μl of CBD mixture per hour.

6. The method of using a transdermal patch to treat a patient with CBD of claim 1, wherein the step of applying the patch to the skin comprises the step of using an at least one microneedle on the patch to puncture the stratum corneum, the viable epidermis, and the dermis, so that when the patch is positioned upon the skin, a tip of the at least one microneedle is positioned within the dermis.

7. The method of using a transdermal patch to treat a patient with CBD of claim 1, wherein the step of allowing the CBD mixture to penetrate the skin is pressure driven.

8. A microneedle device, comprising:
a membrane having an adhesive thereon;
a microneedle substrate adhered to the membrane using the adhesive;
a release layer positioned upon the microneedle device to protect the microneedle substrate;
wherein the microneedle substrate has a plurality of microneedles coupled thereto; and
a CBD mixture comprising CBD oil within liposomes is loaded onto or into the microneedle device.

9. The microneedle device of claim 8, wherein the microneedles are solid and coated with the CBD mixture

10. The microneedle device of claim 8, wherein the microneedles are hollow.

11. The microneedle device of claim 10, wherein the microneedles are connected to a pressure mechanism configured to force CBD mixture out of the needles.

12. The microneedle device of claim 8, wherein the plurality of microneedles comprises microneedle tips configured to dissolve in interstitial body fluid.

13. The microneedle device of claim 8, forming part of a system, the system further comprising a drug container having wells defined therein, the wells configured to hold CBD mixture.

14. The device of claim 8, wherein the plurality of microneedles is at least partially coated with the CBD mixture.

15. A method of using a microneedle device for delivery of CBD, comprising the steps of:
placing a microneedle device of the present disclosure upon the skin of a wearer so to cause at least part of a plurality of microneedles of the microneedle device to enter a dermis of the skin; and
removing the microneedle device from the skin after a CBD mixture enters the interstitial body fluid from the plurality of microneedles, the CBD mixture comprising CBD oil mixed with liposomes.

16. The method of using a microneedle device for delivery of CBD as in claim 15 further comprising the step of removing the microneedle device from a drug container, wherein the drug container comprises wells filled with CBD mixture.

17. The method of using a microneedle device for delivery of CBD as in claim 15, further comprising the step of introducing the CBD mixture into the interstitial body fluid via diffusion from the plurality of microneedles.

18. The method of using a microneedle device for delivery of CBD as in claim 15, wherein the CBD mixture enters the interstitial body fluid from the plurality of microneedles without the use of dermal penetrant enhancers.

19. The method of using a microneedle device for delivery of CBD as in claim 15, further comprising the step of introducing the CBD mixture into the interstitial body fluid at a rate of greater than 0.2 μl per hour.

20. The method of using a microneedle device for delivery of CBD as in claim 15, wherein the CBD mixture comprises liposomes carrying CBD oil; and
the method further comprises the step of allowing the liposome carrying the CBD oil to fuse with bilayers within the patient to deliver the CBD oil.
